(19) United States
(12) Patent Application Publication
(10) Pub. No.: US 2026/0087623 A1
(43) Pub. Date: Mar. 26, 2026

(54) FUNCTIONAL STENOSIS ASSESSMENT FROM VASCULAR IMAGING

(71) Applicant: Boston Scientific Scimed, Inc., Maple Grove, MN (US)

(72) Inventors: Hatice Cinar Akakin, Eden Prairie, MN (US); Qian Li, San Jose, CA (US); Kevin Bloms, Minneapolis, MN (US); Wenguang Li, Los Gatos, CA (US)

(73) Assignee: Boston Scientific Scimed, Inc., Maple Grove, MN (US)

(21) Appl. No.: 19/338,698

(22) Filed: Sep. 24, 2025

Related U.S. Application Data
(60) Provisional application No. 63/699,273, filed on Sep. 26, 2024.

(51) Int. Cl. G06T 7/00; A61B 6/00; A61B 6/50; A61B 8/00; A61B 8/04; G06T 7/12; G06T 17/20

(52) U.S. Cl. CPC G06T 7/0012; A61B 6/507; A61B 6/5217; A61B 8/04; A61B 8/5223; G06T 7/12; G06T 17/20; G06T 2207/20081; G06T 2207/30048; G06T 2207/30104; G06T 2210/41

(57) ABSTRACT

The present disclosure provides to generate ground truth data for training a machine learning (ML) model to infer pressure information (e.g., a pressure curve, a pressure ratio, or the like) for a cardiac artery from border segmentations generated from images of the cardiac artery. The ground truth data can comprise vessel and/or lumen segmentations for several cardiac arteries and associated pressure information for the cardiac arteries. The vessel and/or lumen segmentations can be generated from images from different image modalities (e.g., IVUS, angiographic, CT, etc.). Further, some of the associated pressure information can be based on measured pressure information (e.g., using a pressure sensing catheter) while other associated pressure information can be derived from the vessel and/or lumen border segmentations using numerical analysis techniques (e.g., CFD, or the like).

### CROSS-REFERENCE TO RELATED APPLICATIONS

This patent application claims the benefit of priority under 35 U.S.C. § 119 to U.S. Provisional Patent Application No. 63/699,273, filed Sep. 26, 2024, which is herein incorporated by reference in its entirety.

### TECHNICAL FIELD

The present disclosure generally relates to intravascular imaging systems. Particularly, but not exclusively, the present disclosure relates to deriving a pressure gradient, such as fractional flow reserve (FFR), from intravascular images.

### BACKGROUND

Numerous tools are on the market to treat vascular lesions including plain old balloon angioplasty (POBA), atherectomy, cutting or scoring balloons, high pressure balloons, and percutaneous coronary intervention (PCI). PCI is a non-surgical procedure where a stent is placed inside a vessel at a location of a stricture using a catheter to increase the size of the lumen in the structure. Prior to placing the stent, various physiological assessments of the vessel are made. For example, a pressure guide wire can be inserted into the vessel and pulled back (e.g., through the region of interest) while measuring pressure. From this measured pressure, a pressure gradient curve, or pressure ratio, such as, fractional flow reserve (FFR), can be derived.

It is to be appreciated that acquiring these pressure measurements adds time and cost to a coronary catheterization procedure, which can increase patient risk. As alternative to physical pressure measurements, blood flow and pressure within coronary arteries can be determined through numerical analysis, such as computational fluid dynamics (CFD), applied to the vessel geometry derived from images.

Such an approach involves solving various fundamental equations of fluid dynamics (e.g., the Navier-Stokes equations). However, deriving pressure gradients using such techniques has a very high computational cost in terms of both computing resources needed to carry out the numerical analysis and, in the time required to complete the numerical analysis. As such, deriving pressure within coronary arteries using CFD is not practical for commercial applications.

Thus, there is a need for systems and techniques to derive pressure gradients without the need for physically measuring pressure or deriving the pressure using CFD.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In general, the present disclosure provides systems and techniques to derive pressure gradients, such as, an FFR value, based on vascular images. Specifically, the present disclosure provides that an FFR value can be inferred using a machine learning (ML) model and a series of intravascular images. Of note, the present disclosure provides systems and techniques to train such ML models to infer (or generate) FFR values without paired intravascular images and pressure gradients. It is to be appreciated that collecting enough paired intravascular images and pressure gradients to properly train an ML model is a time-consuming and expensive process. Further, due to various governmental privacy restrictions accessing a large enough volume of such paired training data may not even be possible.

The present disclosure provides to generate paired intravascular images and pressure gradients using numerical analysis applied to three-dimensional (3D) geometries of vessel lumens extracted from image data. These CFD derived pressure gradients and image data sets can be used to augment or supplement the ground truth data real paired images and measured pressure to form a comprehensive training dataset for an ML model.

It is noted that the present disclosure can be applied to train ML models to infer pressure gradients from any of a variety of imaging modalities, such as, angiography, computed tomography (CT) imaging, magnetic resonance imaging, or the like). For example, pressure gradients can be derived using numerical analysis based on three-dimensional (3D) lumen geometries extracted from an image (or series of images) of a vessel and an ML model trained using such derived pressure gradients and the image (or series of images).

In some embodiments, the disclosure can be implemented as a method for forming ground truth data to train a machine learning (ML) model to infer pressure information for a cardiac artery from border segmentations of the cardiac artery. The method can comprise receiving vessel and/or lumen border segmentations for a plurality of cardiac arteries; generating models of the plurality of cardiac arteries from the vessel and/or lumen border segmentations; deriving pressure information for each of the plurality of cardiac arteries based in part on solving a system of equations defining the pressure information using numerical analysis applied to the models of the plurality of cardiac arteries; and adding the vessel and/or lumen border segmentations and the associated derived pressure information to ground truth data for training a machine learning (ML) model.

With some embodiments of the method, generating the model of the cardiac arteries from the vessel and/or lumen border segmentations comprises generating three-dimensional (3D) volumes for each of the cardiac arteries from the vessel and/or lumen border segmentations; generating volume meshes for each of the cardiac arteries from the 3D volumes, wherein each volume mesh comprises a plurality of discrete elements; and solving, for each of the volume meshes, the system of equations for each one of the plurality of discrete elements of the volume mesh.

With some embodiments of the method, the pressure information is a pressure curve defining pressure ratios along a portion of the length of the cardiac artery.

With some embodiments of the method, the pressure curve defines distal pressure (Pd) over proximal pressure (Pa) along the portion of the length of the cardiac artery.

With some embodiments of the method, the vessel and/or lumen border segmentations comprises both vessel and lumen border segmentations.

With some embodiments of the method, receiving the vessel and/or lumen border segmentations for the plurality of cardiac arteries comprises receiving image data associated with each of the cardiac arteries; and generating the vessel and/or lumen border segmentations from the image data.

With some embodiments of the method, generating the vessel and/or lumen border segmentations from the image data comprises applying an image processing algorithm to the image data to identify borders of the vessel and/or lumen of the cardiac arteries.

With some embodiments of the method, the plurality of cardiac arteries are a first plurality of cardiac arteries and the image data comprises image data of a first image modality, and the method further comprise receiving second image data associated with each of a second plurality of cardiac arteries, the second image data comprising image data of a second image modality different than the first image modality; generating vessel and/or lumen border segmentations for each of the second plurality of cardiac arteries from the second image data; generating models of the second plurality of cardiac arteries from the vessel and/or lumen border segmentations; deriving pressure information for each of the second plurality of cardiac arteries based in part on solving the system of equations defining the pressure information using numerical analysis applied to the models of the second plurality of cardiac arteries; and adding the vessel and/or lumen border segmentations or each of the second plurality of cardiac arteries and the associated derived pressure information to the ground truth data.

With some embodiments of the method, the first image modality is intravascular ultrasound (IVUS), optical coherence tomography (OCT), angiographic, magnetic resonance imaging (MRI), or coronary computed tomography angiography (CCTA).

With some embodiments of the method, the second image modality is intravascular ultrasound (IVUS), optical coherence tomography (OCT), angiographic, magnetic resonance imaging (MRI), or coronary computed tomography angiography (CCTA).

With some embodiments, the method further comprises training the ML model with the ground truth data.

With some embodiments of the method, the system of equations is the Navier-Stokes equations.

With some embodiments of the method, the plurality of cardiac arteries are a first plurality of cardiac arteries, and the method further comprises receiving vessel and/or lumen border segmentations for a second plurality of cardiac arteries; receiving pressure information associated with each of the second plurality of cardiac arteries, wherein the pressure information associated with each of the second plurality of cardiac arteries is based on pressure measured with an intravascular pressure measurement device; and adding the vessel and/or lumen border segmentations and the pressure information for the second plurality of cardiac arteries to the ground truth data.

In some embodiments, the disclosure can be implemented as a computing system comprising a processor and memory. The memory can comprise instructions executable by the processor and which when executed, cause the computing system to implement any of the methods outlined herein.

In some embodiments, the disclosure can be implemented as a computer-readable medium comprising instructions that when executed by a processor of a computing system cause the computing system to implement any of the methods outlined herein.

In some embodiments, the disclosure can be implemented as a computing system to train a machine learning (ML) model to infer pressure information for a cardiac artery from border segmentations of the cardiac artery. The computing system can comprise a processor; and memory comprising instructions executable by the processor, which instructions when executed cause the computing system to receive vessel and/or lumen border segmentations for a plurality of cardiac arteries; generate models of the plurality of cardiac arteries from the vessel and/or lumen border segmentations; derive pressure information for each of the plurality of cardiac arteries based in part on solving a system of equations defining the pressure information using numerical analysis applied to the models of the plurality of cardiac arteries; and add the vessel and/or lumen border segmentations and the associated derived pressure information to ground truth data for training a machine learning (ML) model.

With some embodiments of the computing system, when executed the instructions further cause the computing system to generate three-dimensional (3D) volumes for each of the cardiac arteries from the vessel and/or lumen border segmentations; generate volume meshes for each of the cardiac arteries from the 3D volumes, wherein each volume mesh comprises a plurality of discrete elements; and solve, for each of the volume meshes, the system of equations for each one of the plurality of discrete elements of the volume mesh.

With some embodiments of the computing system, the pressure information is a pressure curve defining pressure ratios along a portion of the length of the cardiac artery.

With some embodiments of the computing system, the pressure curve defines distal pressure (Pd) over proximal pressure (Pa) along the portion of the length of the cardiac artery.

With some embodiments of the computing system, the vessel and/or lumen border segmentations comprises both vessel and lumen border segmentations.

With some embodiments of the computing system, when executed the instructions further cause the computing system to receive image data associated with each of the cardiac arteries; and generate the vessel and/or lumen border segmentations from the image data.

With some embodiments of the computing system, when executed the instructions further cause the computing system to apply an image processing algorithm to the image data to identify borders of the vessel and/or lumen of the cardiac arteries.

With some embodiments of the computing system, the plurality of cardiac arteries are a first plurality of cardiac arteries, the image data comprises image data of a first image modality, and when executed the instructions further cause the computing system to receive second image data associated with each of a second plurality of cardiac arteries, the second image data comprising image data of a second image modality different than the first image modality; generate vessel and/or lumen border segmentations for each of the second plurality of cardiac arteries from the second image data; generate models of the second plurality of cardiac arteries from the vessel and/or lumen border segmentations; derive pressure information for each of the second plurality of cardiac arteries based in part on solving the system of equations defining the pressure information using numerical analysis applied to the models of the second plurality of cardiac arteries; and add the vessel and/or lumen border segmentations or each of the second plurality of cardiac arteries and the associated derived pressure information to the ground truth data.

With some embodiments of the computing system, the first image modality is intravascular ultrasound (IVUS), optical coherence tomography (OCT), angiographic, magnetic resonance imaging (MRI), or coronary computed tomography angiography (CCTA).

With some embodiments of the computing system, the second image modality is intravascular ultrasound (IVUS), optical coherence tomography (OCT), angiographic, magnetic resonance imaging (MRI), or coronary computed tomography angiography (CCTA).

With some embodiments of the computing system, the instructions further cause the computing system to train the ML model with the ground truth data.

With some embodiments of the computing system, the system of equations is the Navier-Stokes equations.

With some embodiments of the computing system, the plurality of cardiac arteries are a first plurality of cardiac arteries, and wherein when executed the instructions further cause the computing system to receive vessel and/or lumen border segmentations for a second plurality of cardiac arteries; receive pressure information associated with each of the second plurality of cardiac arteries, wherein the pressure information associated with each of the second plurality of cardiac arteries is based on pressure measured with an intravascular pressure measurement device; and add the vessel and/or lumen border segmentations and the pressure information for the second plurality of cardiac arteries to the ground truth data.

In some embodiments, the disclosure can be implemented as a non-transitory computer-readable storage device. The storage device can comprise instructions that when executed by a processor of a computing system cause the computing system to receive vessel and/or lumen border segmentations for a plurality of cardiac arteries; generate models of the plurality of cardiac arteries from the vessel and/or lumen border segmentations; derive pressure information for each of the plurality of cardiac arteries based in part on solving a system of equations defining the pressure information using numerical analysis applied to the models of the plurality of cardiac arteries; and add the vessel and/or lumen border segmentations and the associated derived pressure information to ground truth data for training a machine learning (ML) model.

With some embodiments of the storage device, when executed the instructions further cause the computing system to generating three-dimensional (3D) volumes for each of the cardiac arteries from the vessel and/or lumen border segmentations; generating volume meshes for each of the cardiac arteries from the 3D volumes, wherein each volume mesh comprises a plurality of discrete elements; and solving, for each of the volume meshes, the system of equations for each one of the plurality of discrete elements of the volume mesh.

With some embodiments of the storage device, the pressure information is a pressure curve defining pressure ratios along a portion of the length of the cardiac artery.

With some embodiments of the storage device, the pressure curve defines distal pressure (Pd) over proximal pressure (Pa) along the portion of the length of the cardiac artery, and wherein the vessel and/or lumen border segmentations comprises both vessel and lumen border segmentations.

### BRIEF DESCRIPTION OF THE DRAWINGS

To easily identify the discussion of any element or act, the most significant digit or digits in a reference number refer to the figure number in which that element is first introduced.

FIG. 1A and FIG. 1B illustrate an example cardiac artery and pressure information for the cardiac artery.

FIG. 2 illustrates a machine learning (ML) model training environment to train an ML model with ground truth data comprising measured and/or derived pressure information.

FIG. 3A and FIG. 3B illustrate example image data for a cardiac artery and pressure information for the cardiac artery that can be derived from the image data.

FIG. 4 illustrates a logic flow to train an ML model to infer pressure information from border segmentations using ground truth data comprising measured and/or derived pressure information.

FIG. 5 illustrates example ground truth data comprising border segmentations from multiple image modalities and a combination of measured and derived pressure information associated with the border segmentations.

FIG. 6 illustrates a computing device configured to generate ground truth data comprising a combination of measured and derived pressure information associated with border segmentations.

FIG. 7 illustrates another logic flow to generate ground truth data comprising a combination of measured and derived pressure information associated with border segmentations.

FIG. 8 illustrates an example imaging system to infer pressure information from border segmentations using an ML model.

FIG. 9 illustrates a computer-readable storage medium.

FIG. 10 illustrates a computing system.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

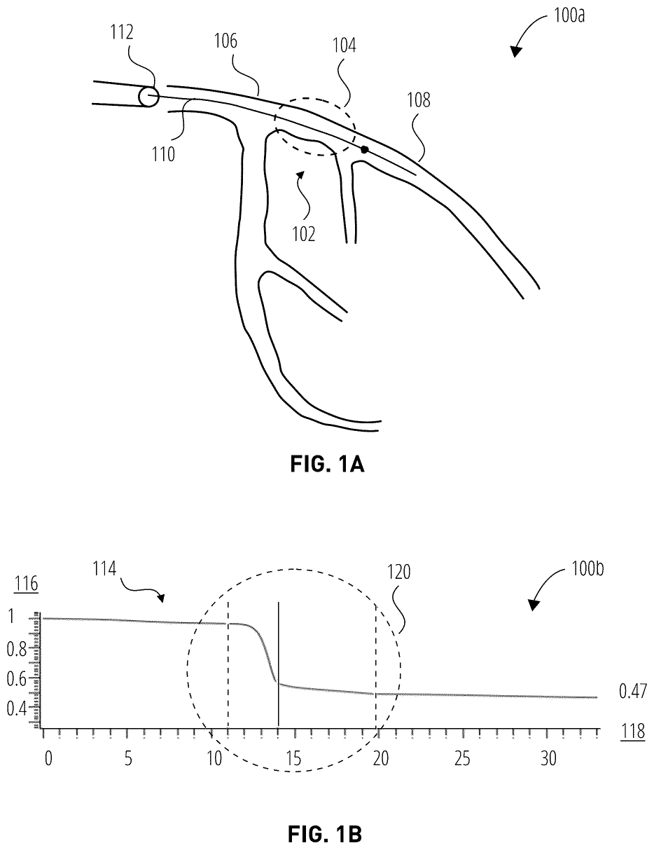

As outlined above, during some medical interventions (e.g., pre-percutaneous coronary intervention (pre-PCI), physicians may desire to assess the pressure drop across a point in an artery. For example, FIG. 1A illustrates an image 100a of an artery 102 having a stenosis 104 (or narrowing of the lumen). Conventionally, the pressure at a proximal point 106 to the stenosis 104 and a distal point 108 to the stenosis 104 would be measured using a pressure measurement device 110 inserted into the artery 102 via a catheter 112. From these measured pressures (e.g., at the proximal point 106 and the distal point 108, or the like), a pressure drop across the stenosis 104 can be visualized.

FIG. 1B illustrates a plot 100b of a pressure ratio curve 114 that can be derived from pressure measured within an artery (e.g., artery 102, or the like). One common pressure ratio is fractional flow reserve (FFR), which is the ratio of the pressure at the distal point 108 (often referred to as (P_{d}) over the pressure at the proximal point 106 (often referred to as Pₐ). In other words, FFR can be expressed as P_{d}/Pₐ. As can be seen from this figure, the pressure ratio curve 114 shows a pressure ratio (e.g., FFR) where the pressure ratio is represented as a ratio between 0 and 1 on the y axis 116 plotted against the distance from the catheter 112 on the x axis 118. The drop in the pressure ratio in region 120 can be associated with the stenosis 104. Often, the severity of the stenosis 104 can be correlated to the pressure drop represented in a plot of the pressure ratio curve like plot 100b.

The present disclosure provides machine learning (ML) training methodologies, and particularly, a methodology for creating training datasets to train ML models to infer a pressure ratio, or a pressure ratio curve from images of arteries in real time (e.g., during an imaging procedure, or the like). This is described in greater detail below. However, prior to describing an example commercial imaging system implementing ML models trained as outlined herein; an example ML training system as well as training methodologies and systems and techniques to generate ground truth training data are described.

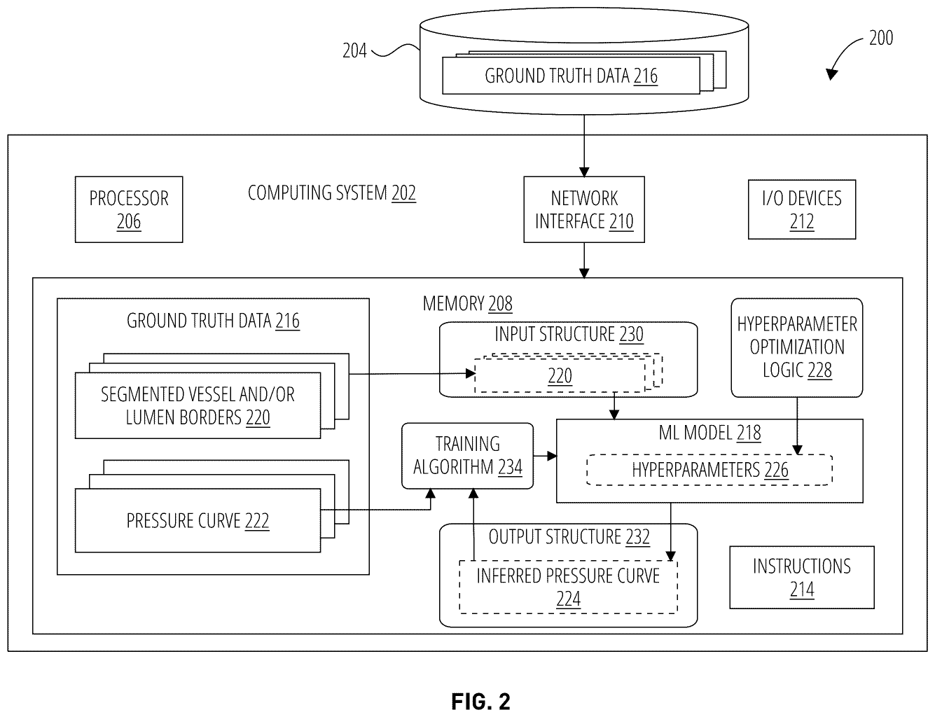

FIG. 2 illustrates an ML training environment 200, according to some embodiments of the present disclosure. In general, ML training environment 200 provides computing resources to train an ML model, or to apply training algorithms such that the ML model may learn relationships between images of arteries and pressure curves for the arteries. With some examples, ML training environment 200 may be implemented to train an ML model to infer a pressure curve from segmented lumen borders for an artery, where the segmented lumen borders can be derived from images of the artery. For example, segmented lumen borders can be generated from intravascular ultrasound (IVUS) images, angiographic images, computed tomography (CT) images, or the like. This is described in greater detail below.

ML training environment 200 includes computing system 202 coupled to an external data storage system 204. In general, computing system 202 can be any of a variety of computing devices. With some examples, computing system 202 can be a specially designed computing system configured to process and train ML models. Computing system 202 can be a workstation, a server, a cloud based computing device, a set of distributed computing resources (e.g., accessibly over a network like the Internet, or the like). External data storage system 204 can be a network accessible data storage device. For example, external data storage system 204 can be a network-attached storage (NAS) device, a storage area network (SAN), data storage accessible via the Internet, or the like. In some examples, external data storage system 204 can be incorporated into computing system 202 (e.g., provisioned as part of the same set of computing resources, or the like).

Computing system 202 can include processor 206, memory 208, network interface 210, and I/O devices 212. Processor 206 may include circuitry or processor logic, such as, for example, any of a variety of commercial processors. In some examples, processor 206 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 206 may include graphics processing circuitry, machine learning (ML) processing circuitry, or other specialized processing circuitry. Further, processor 206 may include dedicated memory, multiple-threaded processing and/or parallel processing capabilities. In some examples, the processor 206 may be an application specific integrated circuit (ASIC) specifically designed for ML training environment 200 and applying ML training algorithms to learn relationships between inputs and outputs as described herein.

Memory 208 may include logic, a portion of which includes arrays of integrated circuits, forming non-volatile memory to persistently store data or a combination of non-volatile memory and volatile memory. It is to be appreciated, that the memory 208 may be based on any of a variety of technologies. In particular, the arrays of integrated circuits included in memory 208 may be arranged to form one or more types of memory, such as, for example, dynamic random access memory (DRAM), NAND memory, NOR memory, or the like. It is noted, while not specifically shown, external data storage system 204 will include arrays of integrated circuits forming non-volatile memory to persistently store data.

Network interface 210 can include logic and/or features to support a communication interface. For example, network interface 210 may include one or more interfaces that operate according to various communication protocols or standards to communicate over direct or network communication links. Direct communications may occur via use of communication protocols or standards described in one or more industry standards (including progenies and variants). For example, network interface 210 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. As another example, network interface 210 can include logic and/or features to enable communication over a variety of wired or wireless network standards. For example, network interface 210 may be arranged to support wired communication protocols or standards (e.g., Ethernet, or the like) or wireless communication protocols or standards (e.g., Wi-Fi, Bluetooth, 5G, or the like).

I/O devices 212 can be any of a variety of devices to receive input and/or provide output. For example, I/O devices 212 can include, a keyboard, a mouse, a joystick, a foot pedal, a display, a touch enabled display, a haptic feedback device, an LED, or the like.

Memory 208 can include instructions 214, ground truth data 216, and ML model 218. During operation, processor 206 can execute instructions 214 to cause computing system 202 to receive (e.g., from external data storage system 204, or the like) ground truth data 216. In some examples, ground truth data 216 can include segmented vessel and/or lumen borders 220 paired with a pressure curve 222.

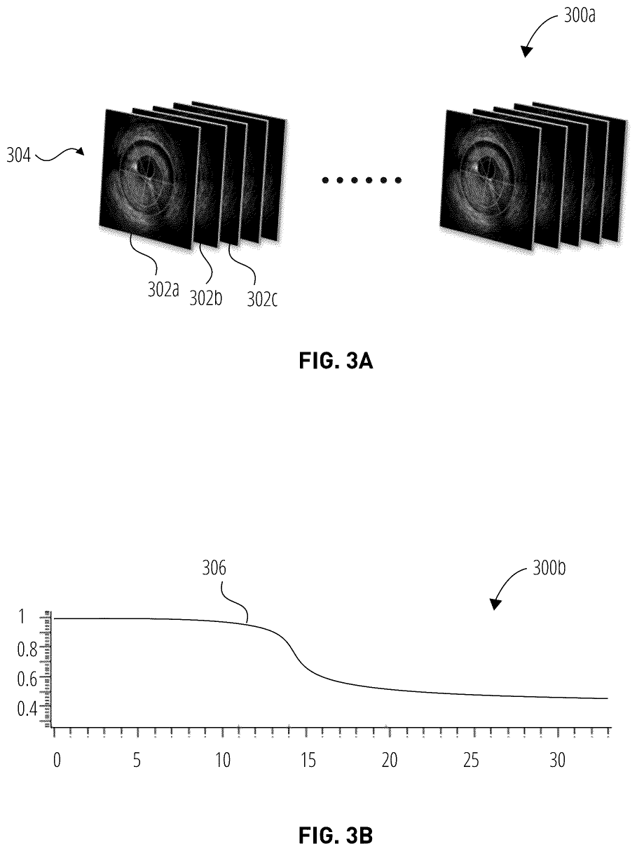

For example, FIG. 3A illustrates a series of images 300a. In this example, the series of images 300a are IVUS images and include several frames 302a, 302b, 302c, etc. The vessel and/or lumen border represented in each frame can be determined. For example, geometric characteristics of the vessel and lumen (e.g., diameter, area, segmented borders, etc.) represented in the series of images 300a can be identified. With some examples, ML training environment 200 is configured to identify these characteristics while in other examples, the characteristics are already identified and included as part of ground truth data 216. For example, FIG. 2 shows ML training environment 200 comprising ground truth data 216 where ground truth data 216 itself comprises several segmented vessel and/or lumen borders 220 paired with a pressure curve 222.

As a specific example, the lumen and/or vessel border can be identified from each frame of series of images 300a. Lumen and vessel border 304 for frame 302a is called out in FIG. 3A. Continuing with this example, segmented vessel and/or lumen borders 220 can comprise indications of lumen and vessel border 304 for each frame of the series of images 300a. As an alternative example, ground truth data 216 can comprise a series of images (e.g., series of images 300a) and processor 206 can execute instructions 214 to derive segmented vessel and/or lumen borders 220 from the series of images. For example, processor 206 can execute instructions 214 to determine segmented vessel and/or lumen borders 220 from a series of images using image processing and geometric image analysis (e.g., object border segmentation, or the like). It is again noted that although the disclosure depicts a series of IVUS series of images 300a, the segmented vessel and/or lumen borders 220 can be derived from any of a variety or combination of two-dimensional (2D) or 3D images. As a practical example, ground truth data 216 could include segmented vessel and/or lumen borders 220 derived from IVUS images, angiographic images, and CT images.

Ground truth data 216 further includes a pressure curve 222 for each set of segmented vessel and/or lumen borders 220. FIG. 3B illustrates plot 300b showing a pressure curve 306 corresponding to the vessel represented in the series of images 300a. With some examples, some of the pressure curves 222 represented in ground truth data 216 can be measured. For example, ground truth data 216 could include several sets of segmented vessel and/or lumen borders 220 and associated pressure curves 222 for each set of segmented vessel and/or lumen borders 220. Some of the sets of segmented vessel and/or lumen borders 220 can be derived from actual images of a vessel captured during a pressure measurement procedure. As such, the pressure curves 222 associated with these sets of segmented vessel and/or lumen borders 220 can be from actual measurements (e.g., using a pressure measurement device 110, or the like).

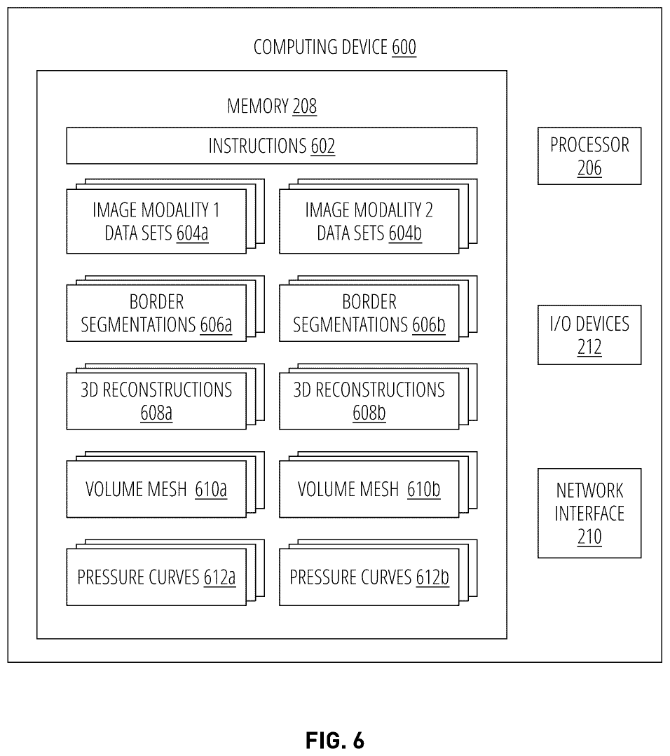

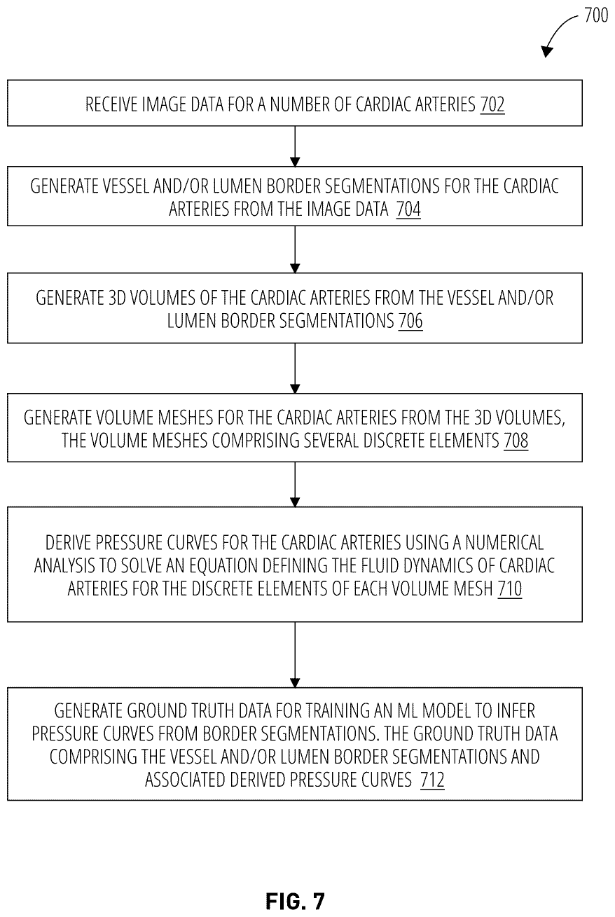

Further, some of the sets of segmented vessel and/or lumen borders 220 can be derived from images of a vessel while the pressure curves 222 associated with these sets of segmented vessel and/or lumen borders 220 can be derived using CFD from the segmented vessel and/or lumen borders 220. An example of this is described later (e.g., with reference to FIG. 6 and FIG. 7).

Returning to FIG. 2 and ML training environment 200, ground truth data 216 can include both measured pressure curves 222 along with associated segmented vessel and/or lumen borders 220 as well as CFD derived pressure curves 222 along with associated segmented vessel and/or lumen borders 220. Further, as noted above, segmented vessel and/or lumen borders 220 can be generated from several different imaging modalities. As such, a comprehensive set of ground truth data 216 can be generated.

During operation, processor 206 may execute instructions 214 to apply segmented vessel and/or lumen borders 220 to train ML model 218 to infer the associated pressure curve 222. Said differently, ML model 218 may be structured to receive segmented vessel and/or lumen borders 220 and input and generate an inferred pressure curve 224 as output. Depending on the application, different types of ML models 218 may be suitable for use. For instance, in the present example, an artificial neural network (ANN) may be particularly well-suited to learning associations between segmented vessel and/or lumen borders 220 and pressure curve 222. Similarity and metric distance learning may also be well-suited to this task, although one of ordinary skill in the art will recognize that different types of ML models 218 may be used, depending on the designer's goals, the resources available, the amount of input data available, etc.

Although not shown in this figure, the underlying structure or architecture of the ML model 218 may comprise several input nodes and several output nodes interconnected by nodes from several hidden layers via an activation function and weights. These characteristics, or structure of the ML model 218, are generally referred to as hyperparameters 226. In some examples, the hyperparameters 226 can be specified by a user of ML training environment 200 while in other examples, processor 206 can execute instructions 214 to apply hyperparameter optimization logic 228 to automatically select the hyperparameters 226. Hyperparameter optimization logic 228 can be based on any known hyperparameter optimization techniques as appropriate to the ML model 218.

The input to ML model 218 can conform to an input structure 230 while the output from the ML model 218 can conform to an output structure 232. For example, segmented vessel and/or lumen borders 220, and as such, input structure 230, can be formatted as any data structure configured to convey indications of border segments, a border area, a border diameter, or the like. Likewise, pressure curve 222, and as such, output structure 232 and inferred pressure curve 224, can be formatted as any data structure configured to convey indications of a curve (e.g., x and y coordinates, or the like).

During training, processor 206 can execute instructions 214 to iteratively apply the segmented vessel and/or lumen borders 220 as input to the ML model 218 and receive inferred pressure curve 224 as output from the ML model 218. Any suitable training algorithm 234 can be utilized to “train” the ML model 218 so that inferred pressure curve 224 is within an acceptable error from pressure curve 222. The example depicted in this figure, may be particularly well-suited to a supervised training algorithm or reinforcement learning technique. In such an example, processor 206 can execute instructions 214 to apply training algorithm 234 to compare the actual model output (e.g., inferred pressure curve 224) with the expected output (e.g., pressure curve 222) and adjust, or “optimize” the hyperparameters 226 (e.g., the weights for connection(s) between nodes, or the like) based on a loss function and/or an optimization function (e.g., gradient descent, or the like). In some examples, the loss function can include one loss component associated with measured pressure curves 222 and another loss component from CFD derived pressure curves 222.

With some examples, some portion (e.g., a first subset) of ground truth data 216 may be used to initially train the ML model 218 while another portion (e.g., a second subset mutually exclusive from the first subset) of ground truth data 216 may be held back and used as a validation or testing data. For example, the first subset of ground truth data 216 can be used to train the ML model 218, whereas the second subset of ground truth data 216 can be used to test the trained ML model 218 to verify that the ML model 218 is able to generalize to “unseen data.” Said differently, the second subset of ground truth data 216 can be applied to ML model 218 to determine whether the inferred pressure curve 224 matches, within an acceptable tolerance, the expected pressure curve 222 for segmented vessel and/or lumen borders 220 not used in training. Completion and/or conclusion of training of ML model 218 can be based on how the ML model 218 is generalized to this validation data.

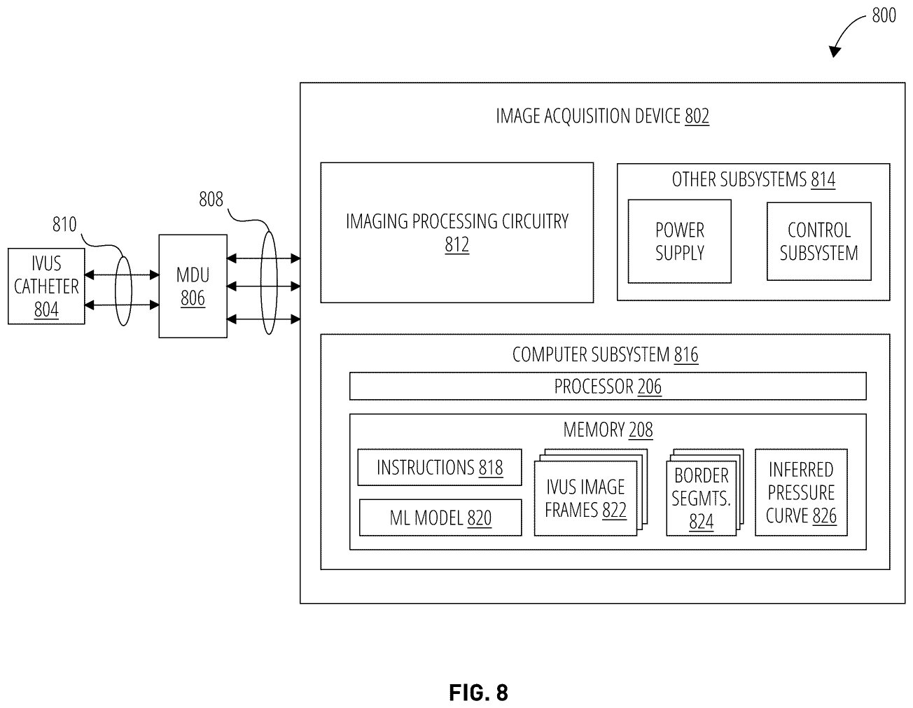

Once the ML model 218 is trained, it may be applied (by the processor 206, by another processor) to infer a pressure curve (e.g., inferred pressure curve 224) from new input data (e.g., vessel and/or lumen border segments derived from images acquired during a cardiac artery imaging procedure). An example of this is provided later (e.g., with reference to FIG. 8).

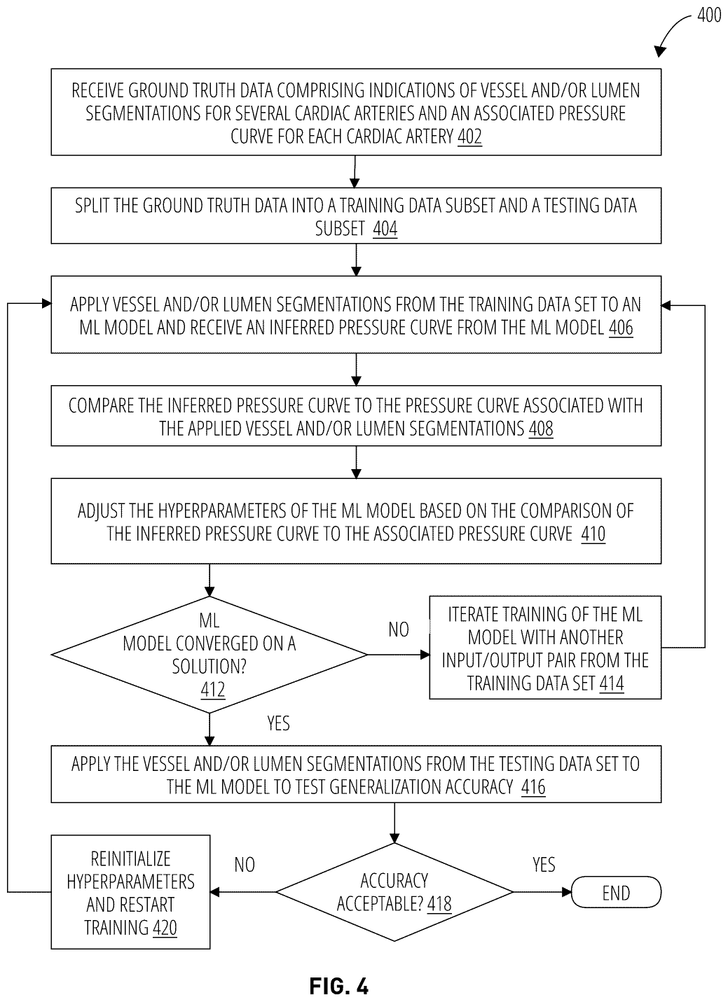

FIG. 4 illustrates a logic flow 400 to train an ML model to infer a pressure curve for a cardiac artery from image data of the artery or vessel and/or lumen border segments derived from the image data. The logic flow 400 can be implemented by ML training environment 200 and will be described with reference to ML training environment 200 for clarity of presentation. However, it is noted that logic flow 400 could also be implemented by any ML training system different than ML training environment 200.

Logic flow 400 can begin at block 402. At block 402 “receive ground truth data comprising indications of vessel and/or lumen segmentations for several cardiac arteries and an associated pressure curve for each cardiac artery” ground truth data comprising indications of vessel and/or lumen segmentations for several cardiac arteries and an associated pressure curve for each cardiac artery can be received. For example, ground truth data 216 comprising sets of segmented vessel and/or lumen borders 220 and associated pressure curves 222 can be received. Processor 206 can execute instructions 214 to received ground truth data 216 from external data storage system 204, or the like. It is noted that the disclosure provides to generate all or portions of ground truth data 216. However, this is described in greater detail below.

Continuing to block 404 “split the ground truth data into a training data subset and a testing data subset” the ground truth data can be split into a first subset of ground truth data to be used for training the ML model and a second subset of ground truth data, mutually exclusive from the first subset, to be used for testing the ML model. For example, processor 206 can execute instructions 214 to select ones of the sets of segmented vessel and/or lumen borders 220 and associated pressure curves 222 from the ground truth data 216 to use as “training data” and while designating the remaining ones as “testing data.” In some examples, 85-95 percent of the ground truth data 216 can be used as training data while the remaining 15-5 percent can be used as testing data.

Continuing to block 406 “apply vessel and/or lumen segmentations from the training data set to an ML model and receive an inferred pressure curve from the ML model” vessel and/or lumen segmentations from the training data set of the ground truth data can be applied to an ML model and an inferred pressure curve received from the ML model. For example, processor 206 can execute instructions 214 to apply segmented vessel and/or lumen borders 220 to ML model 218 and receive inferred pressure curve 224 as output from ML model 218.

Continuing to block 408 “compare the inferred pressure curve to the pressure curve associated with the applied vessel and/or lumen segmentations” the pressure curve associated with the vessel and/or lumen segmentations applied at block 406 can be compared with the inferred pressure curve received at block 406. For example, processor 206 can execute instructions 214 to compare pressure curve 222 to inferred pressure curve 224.

Continuing to block 410 “adjust the hyperparameters of the ML model based on the comparison of the inferred pressure curve to the associated pressure curve” the hyperparameters of the ML model can be adjusted based on the comparison. For example, processor 206 can execute instructions 214 to apply training algorithm 234 to adjust the hyperparameters 226 of the ML model 218 based on the comparison of pressure curve 222 and inferred pressure curve 224.

Continuing to decision block 412 “ML model converged on a solution?” a determination can be made as to whether the ML model has converged on a solution. For example, processor 206 can execute instructions 214 to determine (e.g., based on training algorithm 234, or the like) whether the ML model 218 has converged on a solution (e.g., gradient decent method, or the like). From decision block 412, logic flow 400 can continue to block 414 or block 416. For example, logic flow 400 can continue from decision block 412 to block 414 based on a determination as decision block 412 that the ML model 218 has not converged on a solution while logic flow 400 can continue from decision block 412 to block 416 based on a determination at decision block 412 that the ML model 218 has converged on a solution.

At block 414 “iterate training of the ML model with another input/output pair from the training data set” training of the ML model can continue with another set of segmented vessel and/or lumen borders 220 and an associated pressure curve 222 from the ground truth data 216 (or the training data subset from ground truth data 216). For example, processor 206 can execute instructions 214 to select another set of segmented vessel and/or lumen borders 220 from ground truth data 216 and the logic flow 400 can return to block 406 from block 414 where the selected segmented vessel and/or lumen borders 220 can be applied to the ML model 218.

At block 416 “apply the vessel and/or lumen segmentations from the testing data set to the ML model to test generalization accuracy” vessel and/or lumen segmentations from the testing data set of the ground truth data can be applied to the ML model to “test” the ML model's ability to generalize on unseen data. For example, processor 206 can execute instructions 214 to apply segmented vessel and/or lumen borders 220 from the data in ground truth data 216 reserved as testing data and inferred pressure curves 224 for each set of segmented vessel and/or lumen borders 220 received. Processor 206 can execute instructions 214 to compare the inferred pressure curves 224 with the associated pressure curves 222 to determine an accuracy of the ML model 218.

Continuing to decision block 418 “accuracy acceptable?” a determination can be made as to whether the accuracy of the ML model is acceptable. In some examples, an ML model accuracy is acceptable when it is above a threshold value, such as, 75%, 80%, 85%, 90%, or the like. For example, processor 206 can execute instructions 214 to determine (e.g., based on training algorithm 234, or the like) whether the ML model 218 generalizes (e.g., generates inferred pressure curves 224) based on unseen data and the generated inferred pressure curves 224 have an accuracy (e.g., closeness, or the like) to the associated pressure curves 222 of greater than or equal to an accuracy threshold. From decision block 418, logic flow 400 can end or can continue to block 420. For example, logic flow 400 can continue from decision block 418 to block 420 based on a determination as decision block 418 that the ML model 218 does not have an acceptable accuracy while logic flow 400 can end after decision block 418 based on a determination at decision block 418 that the ML model 218 does have an acceptable accuracy.

At block 420 “reinitialize hyperparameters and restart training” the ML model hyperparameters can be reinitialized and training of the ML model can be restarted. For example, processor 206 can execute instructions 214 to reset and/or reinitialize the hyperparameters 226 of ML model 218 and training of ML model 218 can be restarted. This may be necessary where the ML model 218 has converged on a false minimum solution where the accuracy is insufficient for the application. In such an example, it may be necessary to start training at the beginning. Logic flow 400 can return to block 406 from block 420 where training of the ML model 218 with newly initialized hyperparameters 226 can be restarted.

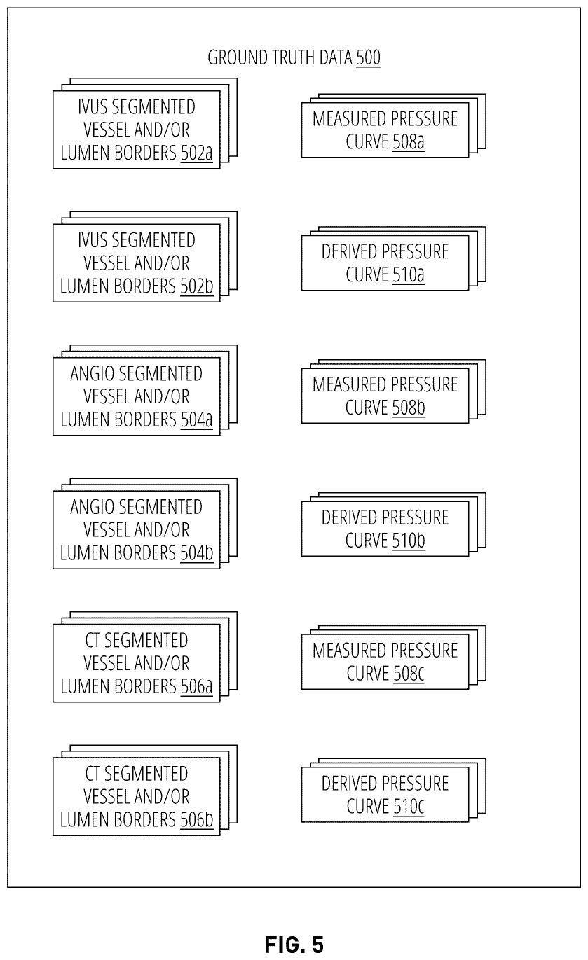

As noted previously, the disclosure provides to augment and/or supplement ground truth data 216 with additional pairs of segmented vessel and/or lumen borders 220 and pressure curve 222, where the pressure curves 222 are generated from the segmented vessel and/or lumen borders 220 using numerical analysis. FIG. 5 illustrates ground truth data 500, which can be formed and used as ground truth data 216. As shown in this figure, ground truth data 500 can comprise several sets of segmented vessel and/or lumen borders 220 determined (e.g., using image analysis as outlined above) from images of vessels captured from multiple different imaging modalities. For example, ground truth data 500 depicts IVUS segmented vessel and/or lumen borders 502a and 502b, which can comprise segmented vessel and/or lumen borders of vessel from IVUS images. Similarly, angio segmented vessel and/or lumen borders 504a and 504b can comprise segmented vessel and/or lumen borders of vessel from angiography images. Further, CT segmented vessel and/or lumen borders 506a and 506b, can comprise segmented vessel and/or lumen borders of vessel from CT images.

It is noted that although only IVUS, angio, and CT images are depicted, ground truth data 216 could be generated based on the disclosure where the segmented vessel and/or lumen borders 220 are generated from images captured using other combinations of image modalities. Further, as outlined above, some of the captured images may have associated pressure curves. For example, the images may have been captured while a pressure was measured and/or in conjunction with measuring a pressure inside the vessel represented in the images. To that end, ground truth data 500 can include IVUS segmented vessel and/or lumen borders 502a that has associated measured pressure curves 508a, angio segmented vessel and/or lumen borders 504a that has associated measured pressure curves 508b, and CT segmented vessel and/or lumen borders 506a that has associated measured pressure curves 508c.

Additionally, ones of the segmented vessel and/or lumen borders 220 may not have an associated pressure curve. For example, these images may comprise representations of a cardiac artery but in which no pressure measurement was taken. As such, the present disclosure provides to generate, using numerical analysis, pressure curves associated with these images, or rather, with these segmented vessel and/or lumen borders 220. For example, derived pressure curves 510a can be generated from IVUS segmented vessel and/or lumen borders 502b, derived pressure curves 510b can be generated from angio segmented vessel and/or lumen borders 504b, and derived pressure curves 510c can be generated from CT segmented vessel and/or lumen borders 506b.

FIG. 6 illustrates a computing device 600, according to some embodiments of the present disclosure configured to generate pressure curves using numerical analysis. With some embodiments, computing device 600 could be implemented in ML training environment 200, such as by computing system 202, and configured to generate pressure curve 222 for ground truth data 216 from segmented vessel and/or lumen borders 220 using numerical analysis as introduced above.

Computing device 600 is depicted with some components of computing system 202 for ease of description of brevity. Like computing system 202, computing device 600 can be any of a variety of computing devices but will in general include processing circuitry and memory. With some examples, the processing circuitry (e.g., processor 206) can be and/or can include specialized processing circuitry for running complex numerical analysis (e.g., CFD computations, or the like).

In computing device 600, memory 208 can include instructions 602, image modality 1 data sets 604a and image modality 2 data sets 604b, vessel and/or lumen border segmentations 606a and vessel and/or lumen border segmentations 606b, 3D reconstructions 608a and 3D reconstructions 608b, volume mesh 610a and volume mesh 610b, and pressure curves 612a and pressure curves 612b. As noted above, the disclosure can be applied to train (and utilize) an ML model to infer a pressure curve from image data, where the image data can be sourced or captured using a variety of imaging modalities. To that end, this figure illustrates computing device 600 configured to generate pressure curves for use in forming ground truth data 216 for multiple image modalities. It is noted that only a first image modality (e.g., image modality 1 data sets 604a) and second image modality (e.g., image modality 2 data sets 604b) are shown in FIG. 6. However, this is done for clarity and brevity, and it is to be appreciated that more than two image modalities could be used. For example, image modality 1 data sets 604a could correspond to IVUS image data sets while image modality 2 data sets 604b could correspond to angiographic image datasets, CT image data sets, or the like.

During operation, processor 206 can execute instructions 602 to receive image modality 1 data sets 604a and image modality 2 data sets 604b and to generate vessel and/or lumen border segmentations 606a and 606b from image modality 1 data sets 604a and image modality 2 data sets 604b, respectively. With some examples, processor 206 can execute instructions 602 to receive vessel and/or lumen border segmentations 606a and vessel and/or border segmentations 606b directly, such as where the border identification and segmentations are generated elsewhere.

Processor 206 can execute instructions 602 to generate 3D reconstructions 608a and 608b from vessel and/or lumen border segmentations 606a and 606b, respectively. For example, processor 206 can execute instructions 602 to reconstruct a 3D model of the vasculature (e.g., cardiac vessels described by the vessel and/or lumen border segmentations 606a and 606b). With some examples, processor 206 can execute instructions 602 to generate 3D reconstructions 608a and 608b by stacking the 2D planes of borders to form a 3D volume representing the vessel lumen.

Processor 206 can execute instructions 602 to generate volume meshes 610a and 610b from 3D reconstructions 608a and 608b, respectively. For example, processor 206 can execute instructions 602 to divide the 3D volumes of reconstructed lumen and vessel walls (e.g., 3D reconstructions 608a and 608b) into sets of small, discrete elements. With some examples, the mesh can have different levels of “quality.” Said differently, the volume meshes 610a and 610b can have elements of a first size for parts of the 3D volume (e.g., lumen wall boundary, curved sections, etc.) while elements of another size, larger than the first size for other parts of the 3D volume can be created (e.g., blood volume, etc.)

Processor 206 can execute instructions 602 to generate pressure curves 612a and 612b from volume meshes 610a and 610b, respectively. In general, processor 206 can execute instructions 602 to solve using numerical analysis (e.g., CFD, finite element analysis, or the like) for each discrete element of the volume meshes 610a and 610b, fundamental equations (e.g., the Navier Stokes equation, or the like) to determine flow through the vessel, and therefore derive pressure curves associated with the vessels.

The Navier-Stokes equations are formed from Newtonian fluidic behavior and Newton's Laws of Motion. In vector form, the Navier-Stokes equation is represented as shown in Equation 1 below.

$$ρ [ \frac{\partial v}{\partial t} + ( v \cdot ∇ ) v ] = - ∇ p + μ {∇}^{2} v \qquad Equation 1$$

In Equation 1, ρ equals the fluid density, p equals the hydrostatic pressure, and μ equals the viscosity coefficient. In general, the right-hand side of Equation 1 represents the change in velocity at a specific location (point) in the fluid (which is not equal to the change in velocity of a particle, as many particles pass through the point over time). Additionally, the left-hand side of the equation represents flow convection, which accounts for transport within the fluid domain (as particles pass through a particular point, they carry with them their inertia and properties to neighboring locations). Processor 206 can execute instructions 602 to solve the partial differential equations of Equation 1, which may require using various boundary conditions. One example is using the zero-dimensional ‘Windkessel-type’ boundary conditions.

Processor 206 can execute instructions 602 to add the vessel and/or lumen border segmentations 606a and 606b as well as the corresponding pressure curves 612a and 612b to ground truth data (e.g., ground truth data 216, ground truth data 500, etc.) for training an ML model as outlined herein.

FIG. 7 illustrates a logic flow 700 to generate ground truth data for training an ML model, according to some embodiments of the present disclosure. The logic flow 700 can be implemented by computing device 600 and will be described with reference to computing device 600 for clarity of presentation. However, it is noted that logic flow 700 could also be implemented by a hemodynamic system different than computing device 600.

Logic flow 700 can begin at block 702. At block 702 “receive image data for a number of cardiac arteries” image data for several cardiac arteries can be received. For example, processor 206 can execute instructions 602 to receive image modality 1 data sets 604a and/or image modality 2 data sets 604b.

Continuing to block 704 “generate vessel and/or lumen border segmentations for the cardiac arteries from the image data” vessel and/or lumen border segmentations for each of the cardiac arteries can be generated from the image data. For example, processor 206 can execute instructions 602 to generate vessel and/or lumen border segmentations 606a and 606b from image modality 1 data sets 604a and 604b. It is noted that with some embodiments, block 704 may be omitted and processor 206 can execute instructions 602 to receive image modality 1 data sets 604a and image modality 2 data sets 604b as well as vessel and/or lumen border segmentations 606a and 606b. Alternatively, processor 206 can execute instructions 602 to receive just vessel and/or lumen border segmentations 606a and 606b at block 702.

Continuing to block 706 “generate 3D volumes of the cardiac arteries from the vessel and/or lumen border segmentations” 3D volumes of the cardiac arteries can be generated from the vessel and/or lumen segmentations. For example, processor 206 can execute instructions 602 to form 3D reconstructions 608a and 608b (e.g., 3D volumes) based in part on stacking the 2D border segmentations represented by the vessel and/or lumen border segmentations 606a and 606b for each cardiac artery.

Continuing to block 708 “generate volume meshes for the cardiac arteries from the 3D volumes, the volume meshes comprising several discrete elements” volume meshes representing each cardiac artery can be formed from the 3D volumes. For example, processor 206 can execute instructions 602 to generate volume meshes 610a and 610b from 3D reconstructions 608a and 608b based on discretizing the 3D volume into multiple elements.

Continuing to block 710 “derive pressure curves for the cardiac arteries using a numerical analysis to solve an equation defining the fluid dynamics of cardiac arteries for the discrete elements of each volume mesh” pressure curves for each cardiac artery can be derived from the volume meshes using numerical analysis to solve an equation defining the fluid dynamics of cardiac arteries. For example, processor 206 can execute instructions 602 to derive pressure curves 612a and 612b from volume meshes 610a and 610b by solving (e.g., with CFD, FEA, etc.) a set of equations that defines the fluid dynamics of cardiac arteries.

Continuing to block 712 “generate ground truth data for training an ML model to infer pressure curves from border segmentations, the ground truth data comprising the vessel and/or lumen border segmentations and associated derived pressure curves” ground truth data can be formed, or supplemented, with the vessel and/or lumen border segmentations and associated pressure curves derived as block 710. For example, processor 206 can execute instructions 602 to add vessel and/or lumen border segmentations 606a and 606b as well as pressure curves 612a and 612b to ground truth data 216. As described herein, this ground truth data (e.g., ground truth data 216, ground truth data 500, etc.) can be used to train an ML model (e.g., ML model 218, or the like) to infer pressure curves from border segmentations.

FIG. 8 illustrates an example imaging system 800, in the form an IVUS imaging system. The imaging system 800 can be configured to generate a pressure curve and/or pressure ratio using an ML model trained as outlined herein. It is noted that although the imaging system 800 depicts an IVUS system, the disclosure could be applied to other imaging modalities (e.g., angiographic, CT, etc.). Imaging system 800 includes an image acquisition device 802, an IVUS catheter 804, and a motor drive unit (MDU) 806.

The image acquisition device 802 is coupled to the IVUS catheter 804 via the MDU 806. In particular, the image acquisition device 802 is coupled to the MDU 806 via the MDU bus 808 while the MDU 806 is coupled to the IVUS catheter 804 via the catheter bus 810. In some embodiments, the MDU bus 808 and the catheter bus 810 can be transmission lines (or other conductors) arranged to convey signals between the various components. For example, the MDU bus 808 and catheter bus 810 can be arranged to transmit radio frequency signals (e.g., control signals, ultrasound pulse generation signals, ultrasound signals, or the like) between the indicated components of the imaging system 800. The IVUS catheter 804 can be a catheter configured for insertion into a body lumen such as a cardiac artery with imaging (e.g., ultrasound, or the like) transducers on a distal end.

In general, the image acquisition device 802 is configured to control the MDU 806 and receive signals from the IVUS catheter 804, via the MDU 806. Further, the image acquisition device 802 is configured to process the received signals to generate IVUS images and can include display components to display the images and/or information derived from the images for a user.

The image acquisition device 802 includes an imaging processing circuitry 812, computer subsystem 816, and other subsystems 814. Imaging processing circuitry 812 can be configured to process electrical signals received from IVUS catheter 804 and can include, for example, circuitry to generate IVUS image frames from these electrical signals. The other subsystems 814 can include any other systems, components, or subsystems for image acquisition device 802, such as power supply circuitry, control circuitry, or the like.

Computer subsystem 816 is depicted with some components of computing system 202 for ease of description of brevity. Like computing system 202, computer subsystem 816 can be any of a variety of computing devices but will in general include processing circuitry and memory. With some examples, the processing circuitry (e.g., processor 206) can be and/or can include specialized processing circuitry designed for an IVUS console.

In computer subsystem 816, memory 208 can include instructions 818, ML model 820, IVUS image frames 822, vessel and/or lumen border segmentations 824, and inferred pressure curve 826. During operation, processor 206 can execute instructions 818 to receive IVUS image frames 822 from imaging processing circuitry 812 where IVUS image frames 822 corresponds to frames of ultrasound images captured by IVUS catheter 804. Further, processor 206 can execute instructions 818 to generate vessel and/or lumen border segmentations 824 from IVUS image frames 822 and to apply vessel and/or lumen border segmentations 824 as input to ML model 820 to generate inferred pressure curve 826.

In such a manner, imaging system 800 provides an improvement over conventional systems and techniques in that a pressure curve (e.g., pressure curve 222) can be derived or inferred from non-invasive techniques (e.g., without inserting a pressure sensing device into the patient's vasculature). Further, the imaging system 800 provides an improvement over conventional system in that the pressure curve can be inferred without necessitating complex and resource intensive computational tasks like numerical analysis.

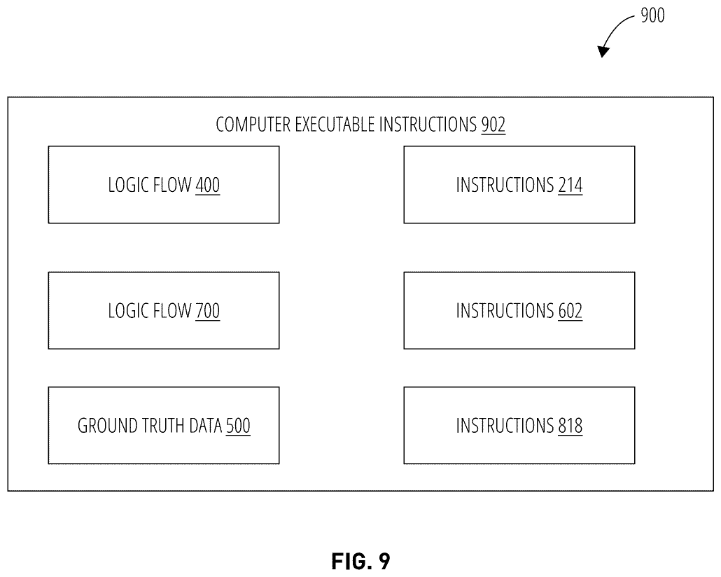

FIG. 9 illustrates computer-readable storage medium 900. Computer-readable storage medium 900 may comprise any non-transitory computer-readable storage medium or machine-readable storage medium, such as an optical, magnetic or semiconductor storage medium. In various embodiments, computer-readable storage medium 900 may comprise an article of manufacture. In some embodiments, computer-readable storage medium 900 may store computer executable instructions 902 with which circuitry (e.g., processor 206, or the like) can execute. For example, computer executable instructions 902 can include instructions to implement operations described with respect to logic flow 400 depicted in FIG. 4, logic flow 700 depicted in FIG. 7, ground truth data 500 depicted in FIG. 5, instructions 214 depicted in FIG. 2, instructions 602 depicted in FIG. 6, and instructions 818 depicted in FIG. 8. Examples of computer-readable storage medium 900 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 902 may include any suitable type of code, such as source code, compiled code, interpreted code, executable code, static code, dynamic code, object-oriented code, visual code, and the like.

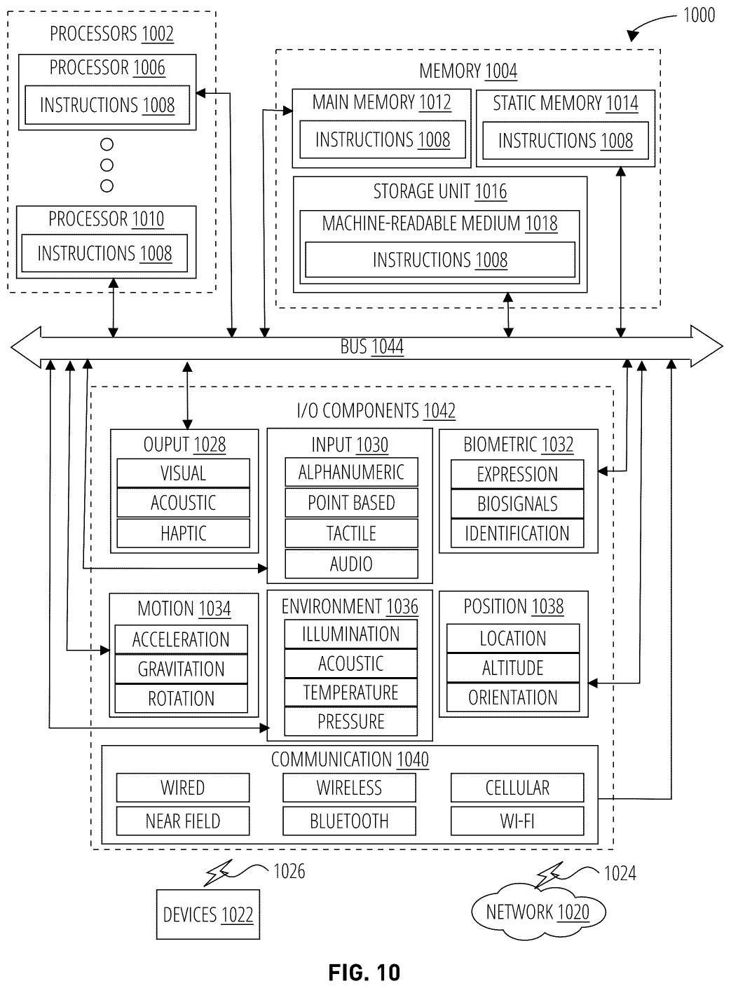

FIG. 10 illustrates a diagrammatic representation of a machine 1000 in the form of a computer system within which a set of instructions may be executed for causing the machine to perform any one or more of the methodologies discussed herein. More specifically, FIG. 10 shows a diagrammatic representation of the machine 1000 in the example form of a computer system, within which instructions 1008 (e.g., software, a program, an application, an applet, an app, or other executable code) for causing the machine 1000 to perform any one or more of the methodologies discussed herein may be executed. For example, the instructions 1008 may cause the machine 1000 to execute logic flow 400 of FIG. 4 and/or logic flow 700 of FIG. 7 to generate ground truth data and/or train an ML model as outlined herein.

The instructions 1008 transform the general, non-programmed machine 1000 into a particular machine 1000 programmed to carry out the described and illustrated functions in a specific manner. In alternative embodiments, the machine 1000 operates as a standalone device or may be coupled (e.g., networked) to other machines. In a networked deployment, the machine 1000 may operate in the capacity of a server machine or a client machine in a server-client network environment, or as a peer machine in a peer-to-peer (or distributed) network environment. The machine 1000 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1008, sequentially or otherwise, that specify actions to be taken by the machine 1000. Further, while only a single machine 1000 is illustrated, the term “machine” shall also be taken to include a collection of machines 1000 that individually or jointly execute the instructions 1008 to perform any one or more of the methodologies discussed herein.

The machine 1000 may include processors 1002, memory 1004, and I/O components 1042, which may be configured to communicate with each other such as via a bus 1044. In an example embodiment, the processors 1002 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1006 and a processor 1010 that may execute the instructions 1008. The term “processor” is intended to include multi-core processors that may comprise two or more independent processors (sometimes referred to as “cores”) that may execute instructions contemporaneously. Although FIG. 10 shows multiple processors 1002, the machine 1000 may include a single processor with a single core, a single processor with multiple cores (e.g., a multi-core processor), multiple processors with a single core, multiple processors with multiples cores, or any combination thereof.

The memory 1004 may include a main memory 1012, a static memory 1014, and a storage unit 1016, both accessible to the processors 1002 such as via the bus 1044. The main memory 1004, the static memory 1014, and storage unit 1016 store the instructions 1008 embodying any one or more of the methodologies or functions described herein. The instructions 1008 may also reside, completely or partially, within the main memory 1012, within the static memory 1014, within machine-readable medium 1018 within the storage unit 1016, within at least one of the processors 1002 (e.g., within the processor's cache memory), or any suitable combination thereof, during execution thereof by the machine 1000.

The I/O components 1042 may include a wide variety of components to receive input, provide output, produce output, transmit information, exchange information, capture measurements, and so on. The specific I/O components 1042 that are included in a particular machine will depend on the type of machine. For example, portable machines such as mobile phones will likely include a touch input device or other such input mechanisms, while a headless server machine will likely not include such a touch input device. It will be appreciated that the I/O components 1042 may include many other components that are not shown in FIG. 10. The I/O components 1042 are grouped according to functionality merely for simplifying the following discussion and the grouping is in no way limiting. In various example embodiments, the I/O components 1042 may include output components 1028 and input components 1030. The output components 1028 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1030 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1042 may include biometric components 1032, motion components 1034, environmental components 1036, or position components 1038, among a wide array of other components. For example, the biometric components 1032 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1034 may include acceleration sensor components (e.g., accelerometer), gravitation sensor components, rotation sensor components (e.g., gyroscope), and so forth. The environmental components 1036 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1038 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1042 may include communication components 1040 operable to couple the machine 1000 to a network 1020 or devices 1022 via a coupling 1024 and a coupling 1026, respectively. For example, the communication components 1040 may include a network interface component or another suitable device to interface with the network 1020. In further examples, the communication components 1040 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1022 may be another machine or any of a wide variety of peripheral devices (e.g., a peripheral device coupled via a USB).

Moreover, the communication components 1040 may detect identifiers or include components operable to detect identifiers. For example, the communication components 1040 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1040, such as location via Internet Protocol (IP) geolocation, location via Wi-Fi® signal triangulation, location via detecting an NFC beacon signal that may indicate a particular location, and so forth.

The various memories (i.e., memory 1004, main memory 1012, static memory 1014, and/or memory of the processors 1002) and/or storage unit 1016 may store one or more sets of instructions and data structures (e.g., software) embodying or utilized by any one or more of the methodologies or functions described herein. These instructions (e.g., the instructions 1008), when executed by processors 1002, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1020 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1020 or a portion of the network 1020 may include a wireless or cellular network, and the coupling 1024 may be a Code Division Multiple Access (CDMA) connection, a Global System for Mobile communications (GSM) connection, or another type of cellular or wireless coupling. In this example, the coupling 1024 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1008 may be transmitted or received over the network 1020 using a transmission medium via a network interface device (e.g., a network interface component included in the communication components 1040) and utilizing any one of several well-known transfer protocols (e.g., hypertext transfer protocol (HTTP)). Similarly, the instructions 1008 may be transmitted or received using a transmission medium via the coupling 1026 (e.g., a peer-to-peer coupling) to the devices 1022. The terms “transmission medium” and “signal medium” mean the same thing and may be used interchangeably in this disclosure. The terms “transmission medium” and “signal medium” shall be taken to include any intangible medium that can store, encoding, or carrying the instructions 1008 for execution by the machine 1000, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A computing system to train a machine learning (ML) model to infer pressure information for a cardiac artery from border segmentations of the cardiac artery, comprising:
a processor; and
memory comprising instructions executable by the processor, which instructions when executed cause the computing system to:
receive vessel and/or lumen border segmentations for a plurality of cardiac arteries;
generate models of the plurality of cardiac arteries from the vessel and/or lumen border segmentations;
derive pressure information for each of the plurality of cardiac arteries based in part on solving a system of equations defining the pressure information using numerical analysis applied to the models of the plurality of cardiac arteries; and
add the vessel and/or lumen border segmentations and the associated derived pressure information to ground truth data for training a machine learning (ML) model.

2. The computing system of claim 1, wherein when executed the instructions further cause the computing system to:
generate three-dimensional (3D) volumes for each of the cardiac arteries from the vessel and/or lumen border segmentations;
generate volume meshes for each of the cardiac arteries from the 3D volumes, wherein each volume mesh comprises a plurality of discrete elements; and
solve, for each of the volume meshes, the system of equations for each one of the plurality of discrete elements of the volume mesh.

3. The computing system of claim 1, wherein the pressure information is a pressure curve defining pressure ratios along a portion of the length of the cardiac artery.

4. The computing system of claim 3, wherein the pressure curve defines distal pressure (Pd) over proximal pressure (Pa) along the portion of the length of the cardiac artery.

5. The computing system of claim 1, wherein the vessel and/or lumen border segmentations comprises both vessel and lumen border segmentations.

6. The computing system of claim 1, wherein when executed the instructions further cause the computing system to:
receive image data associated with each of the cardiac arteries; and
generate the vessel and/or lumen border segmentations from the image data.

7. The computing system of claim 6, wherein when executed the instructions further cause the computing system to apply an image processing algorithm to the image data to identify borders of the vessel and/or lumen of the cardiac arteries.

8. The computing system of claim 6, wherein the plurality of cardiac arteries are a first plurality of cardiac arteries, wherein the image data comprises image data of a first image modality, and wherein when executed the instructions further cause the computing system to:
receive second image data associated with each of a second plurality of cardiac arteries, the second image data comprising image data of a second image modality different than the first image modality;
generate vessel and/or lumen border segmentations for each of the second plurality of cardiac arteries from the second image data;
generate models of the second plurality of cardiac arteries from the vessel and/or lumen border segmentations;
derive pressure information for each of the second plurality of cardiac arteries based in part on solving the system of equations defining the pressure information using numerical analysis applied to the models of the second plurality of cardiac arteries; and
add the vessel and/or lumen border segmentations or each of the second plurality of cardiac arteries and the associated derived pressure information to the ground truth data.

9. The computing system of claim 8, wherein the first image modality is intravascular ultrasound (IVUS), optical coherence tomography (OCT), angiographic, magnetic resonance imaging (MRI), or coronary computed tomography angiography (CCTA).

10. The computing system of claim 8, wherein the second image modality is intravascular ultrasound (IVUS), optical coherence tomography (OCT), angiographic, magnetic resonance imaging (MRI), or coronary computed tomography angiography (CCTA).

11. The computing system of claim 1, wherein when executed the instructions further cause the computing system to train the ML model with the ground truth data.

12. The computing system of claim 1, wherein the system of equations is the Navier-Stokes equations.

13. The computing system of claim 1, wherein the plurality of cardiac arteries are a first plurality of cardiac arteries, and wherein when executed the instructions further cause the computing system to:
receive vessel and/or lumen border segmentations for a second plurality of cardiac arteries;
receive pressure information associated with each of the second plurality of cardiac arteries, wherein the pressure information associated with each of the second plurality of cardiac arteries is based on pressure measured with an intravascular pressure measurement device; and
add the vessel and/or lumen border segmentations and the pressure information for the second plurality of cardiac arteries to the ground truth data.

14. A non-transitory computer-readable storage device, comprising instructions that when executed by a processor of a computing system cause the computing system to:
receive vessel and/or lumen border segmentations for a plurality of cardiac arteries;
generate models of the plurality of cardiac arteries from the vessel and/or lumen border segmentations;
derive pressure information for each of the plurality of cardiac arteries based in part on solving a system of equations defining the pressure information using numerical analysis applied to the models of the plurality of cardiac arteries; and
add the vessel and/or lumen border segmentations and the associated derived pressure information to ground truth data for training a machine learning (ML) model.

15. The non-transitory computer-readable storage device of claim 14, wherein when executed the instructions further cause the computing system to:
generating three-dimensional (3D) volumes for each of the cardiac arteries from the vessel and/or lumen border segmentations;
generating volume meshes for each of the cardiac arteries from the 3D volumes, wherein each volume mesh comprises a plurality of discrete elements; and
solving, for each of the volume meshes, the system of equations for each one of the plurality of discrete elements of the volume mesh.

16. The non-transitory computer-readable storage device of claim 15, wherein the pressure information is a pressure curve defining pressure ratios along a portion of the length of the cardiac artery.

17. The non-transitory computer-readable storage device of claim 16, wherein the pressure curve defines distal pressure (Pd) over proximal pressure (Pa) along the portion of the length of the cardiac artery, and wherein the vessel and/or lumen border segmentations comprises both vessel and lumen border segmentations.

18. A method for forming ground truth data to train a machine learning (ML) model to infer pressure information for a cardiac artery from border segmentations of the cardiac artery, comprising:
receiving vessel and/or lumen border segmentations for a plurality of cardiac arteries;
generating models of the plurality of cardiac arteries from the vessel and/or lumen border segmentations;
deriving pressure information for each of the plurality of cardiac arteries based in part on solving a system of equations defining the pressure information using numerical analysis applied to the models of the plurality of cardiac arteries; and
adding the vessel and/or lumen border segmentations and the associated derived pressure information to ground truth data for training a machine learning (ML) model.

19. The method of claim 18, wherein receiving the vessel and/or lumen border segmentations for the plurality of cardiac arteries comprises:
receiving image data associated with each of the cardiac arteries; and
generating the vessel and/or lumen border segmentations from the image data.

20. The method of claim 19, wherein the plurality of cardiac arteries are a first plurality of cardiac arteries and the image data comprises image data of a first image modality, the method further comprising:
receiving second image data associated with each of a second plurality of cardiac arteries, the second image data comprising image data of a second image modality different than the first image modality;
generating vessel and/or lumen border segmentations for each of the second plurality of cardiac arteries from the second image data;
generating models of the second plurality of cardiac arteries from the vessel and/or lumen border segmentations;
deriving pressure information for each of the second plurality of cardiac arteries based in part on solving the system of equations defining the pressure information using numerical analysis applied to the models of the second plurality of cardiac arteries; and
adding the vessel and/or lumen border segmentations or each of the second plurality of cardiac arteries and the associated derived pressure information to the ground truth data.
